(19) United States
(12) Patent Application Publication
(10) Pub. No.: US 2012/0157886 A1
(43) Pub. Date: Jun. 21, 2012

(54) Mechanomyography Signal Input Device, Human-Machine Operating System and Identification Method Thereof

(75) Inventors: Hian-Kun Tenn, Kaohsiung City (TW); Jiun-Sheng Li, Tainan City (TW); Chia-Chao Chung, Zhubei City (TW)

(73) Assignee: Industrial Technology Research Institute, Hsinchu (TW)

(21) Appl. No.: 13/112,274

(22) Filed: May 20, 2011

(30) Foreign Application Priority Data
Dec. 17, 2010 (TW) 99144565

(51) Int. Cl. A61B 5/11; A61B 5/103

(52) U.S. Cl. 600/595; 600/587

(57) ABSTRACT

A mechanomyography (MMG) signal input device, a human-machine operating system and an identification method thereof are provided. The system includes a mechanomyography (MMG) signal input device, a signal processing unit, a motion database and a calculating unit. The MMG signal input device is mounted on a measuring portion of a testing body, wherein the measuring portion has a plurality of muscle groups. The signal processing unit is used for receiving the MMG signals of the muscle groups, and performing integration and pre-processing on the MMG signals to obtain a processed signal. The motion database is used for storing a motion mode. The calculating unit is used for performing signal intensity computation, data segmentation, feature vector calculation, and testing body's motion recognition, and outputting a corresponding control signal according to the result of recognition.

This application claims the benefit of Taiwan application Serial No. 99144565, filed Dec. 17, 2010, the subject matter of which is incorporated herein by reference.

### BACKGROUND

1. Technical Field

The disclosure relates in general to an input device, a human-machine operating system and an identification method thereof, and more particularly to a mechanomyography (MMG) signal input device, a human-machine operating system and an identification method thereof.

2. Description of the Related Art

In the modern society where medicare is so advanced and convenient, there are still people being handicapped in their limbs due to congenital reasons or post-natal accidents. No matter people are handicapped in their upper or their lower limbs, the impact is tremendous. For example, if people are amputated by their foot or leg, they would have mobility problem and have to rely on the prosthetics or a wheelchair. If people are amputated by their hand or their arm, they would be deprived of basic hand movements, and their daily life would be greatly affected. Despite the prosthetics help to restore the outlook of their limbs and enable the user to use simple arm movements, the prosthetics cannot achieve delicate finger movements. Thus, those people who are unable to operate a device with their fingers due to hand handicap or other restrictions would not be able to use the electronic devices whose input relies on fingers, and a feasible solution must be provided.

### SUMMARY

The disclosure is directed to a mechanomyography (MMG) signal input device, a human-machine operating system and an identification method thereof enabling the user to input signals more conveniently.

The present disclosure provides an MMG signal input device. The MMG signal input device includes a circular body and a plurality of mechanomyography sensing elements. The circular body has a plurality of elastic segments and fixed segments interlaced and sequentially connected into one piece. The lengths of the elastic segments are adjustable, so that the circular body is mounted on a measuring portion of a testing body. The fixed segments respectively have an element embedding surface, and the measuring portion has a plurality of muscle groups. A plurality of mechanomyography sensing elements are disposed on the element embedding surfaces for substantially contacting the measuring portion to measure the MMG signals of the muscle groups respectively.

The present further provides a human-machine operating system. The human-machine operating system includes an MMG signal input device, a signal processing unit, a motion database and a calculating unit. The MMG signal input device is mounted on a measuring portion of a testing body, wherein the measuring portion has a plurality of muscle groups. The signal processing unit is used for receiving the MMG signals of the muscle groups, and performing integration and pre-processing on the MMG signals to obtain a processed signal. The motion database is sued for storing a motion mode. The calculating unit is used for receiving the processed signal and performing signal intensity computation and data segmentation to obtain a segment data, performing feature vector calculation on the segment data to obtain a feature vector data, and performing motion recognition on the testing body according to the feature vector data and the motion mode to output a corresponding control signal.

The present disclosure further provides an MMG signal identification method. The method includes the following steps. A plurality of MMG signals are received, wherein the MMG signals are generated when a plurality of muscle groups of a testing body stretch or contract. Signal integration and pre-processing are performed on the MMG signals to obtain a processed signal. Signal intensity computation and data segmentation are performed on the processed signal to obtain a segment data, and process of feature vector calculation is performed on the segment data to obtain a feature vector data. Motion recognition is performed on a testing body according to the feature vector data and a motion mode. A control signal is outputted according to the result of the motion recognition.

The disclosure will become better understood with regard to the following detailed description of the non-limiting embodiment(s). The following description is made with reference to the accompanying drawings.

### BRIEF DESCRIPTION OF THE DRAWINGS

FIG. 1 shows a cross-sectional view of an MMG signal input device according to an embodiment.

FIG. 2 shows a system architecture of an MMG signal according to an embodiment.

FIG. 3 shows a measurement diagram of an MMG signal in tri-axial directions according to an embodiment.

FIG. 4 shows a processing flowchart of an MMG signal according to an embodiment.

FIG. 5 shows a circuit block diagram of a human-machine operating system according to an embodiment.

### DETAILED DESCRIPTION

According to the mechanomyography (MMG) signal input device, the human-machine operating system and the identification method thereof disclosed in an embodiment of the disclosure, a movement generated when the muscle groups of the limbs collaboratively stretch or contract is used as an input signal, and the feature vector is calculated and the movement corresponding to the MMG signals of the muscle groups is recognized through signal processing so as to output a control signal. In the present embodiment of the disclosure, the user can control the human-machine operating system to output a control signal according to the signals detected by the MMG signal input device to replace the conventional finger signal input method which is used in such as a remote controller, a direction controller, a cursor controller, a limb rehabitation apparatus, a hand-free game player and a movement training machine.

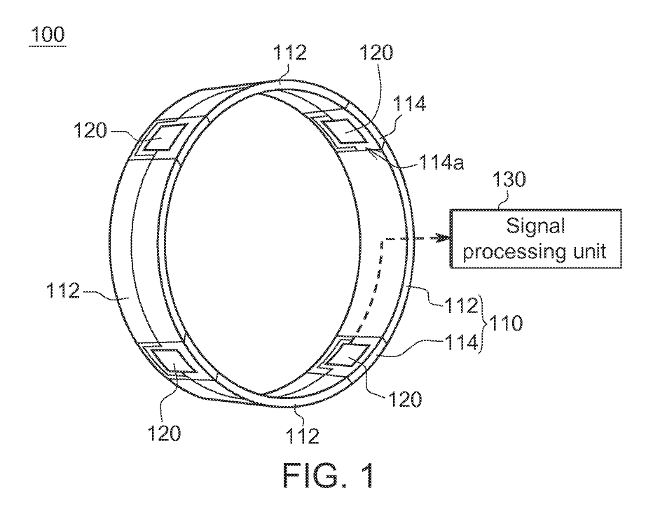

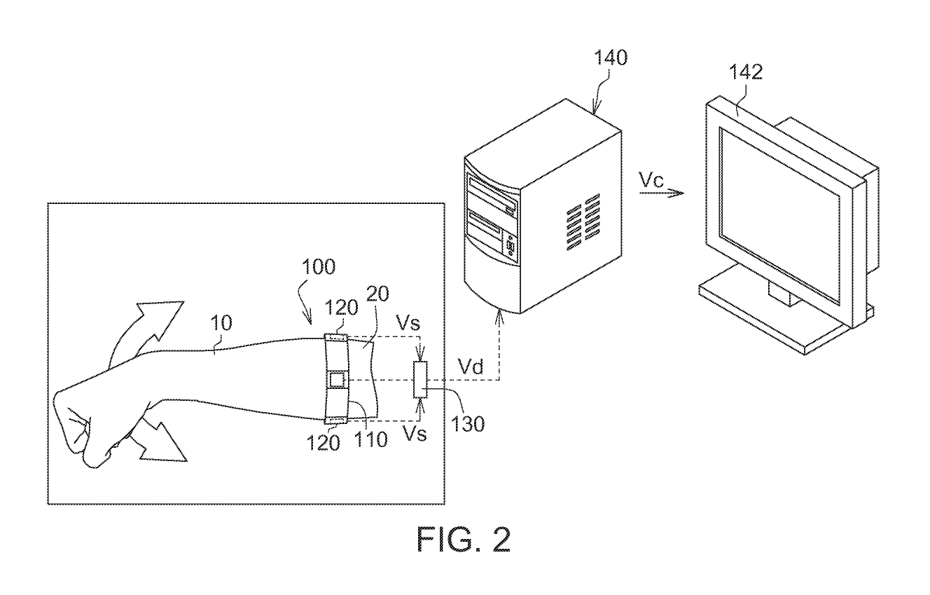

Referring to FIGS. 1 and 2. FIG. 1 shows a cross-sectional view of an MMG signal input device according to an embodiment. FIG. 2 shows a system architecture of an MMG signal according to an embodiment. The MMG signal input device 100 includes a circular body 110 and a plurality of mechanomyography sensing elements 120. The circular body 110 has a plurality of elastic segments 112 and fixed segments 114 which are interlaced and sequentially connected into one piece, wherein the lengths of the elastic segments 112 are adjustable, so that the circular body 110 is mounted on a measuring portion 20 of a testing body 10. The measuring portion 20 has a plurality of muscle groups. In an embodiment, the measuring portion 20 of the testing body 10 is such as the user's upper limb or lower limb, and the muscle groups collaboratively stretch or contract to generate an upper limb movement or a lower limb movement. The elastic segments 112 are made from an elastic material (such as elastic fiber, woven cloth, and silicon) or a combination of chains and loops whose lengths are adjustable. Thus, the lengths of the elastic segments 112 can be adjusted according to the position and size of the measuring portion 20, so that the circular body 110 will not become loose or come off easily after having been mounted on the measuring portion 20 of the testing body 10.

In addition, the fixed segments 114 are disposed between two elastic segments 112. Referring to FIG. 1, the number of the fixed segments 114 can be two or above. In an embodiment, four fixed segments 114 are arranged in different directions (such as facing upward, downward, leftward and rightward) at an equal distance. The number of the fixed segments 114 is not limited to four and the space arrangement is not limited to be at equal distance. The number and space arrangement of the fixed segments 114 can be adjusted according to the type, the number of movements to be determined and the measuring portion. Thus, the circular body 110 of the present embodiment is not limited to have the elastic segments 112 and the fixed segments 114 with fixed numbers, and the number and corresponding directions of the fixed segments 114 can be determined according to the size of the measuring portion 20 and the number of the muscle groups.

In an embodiment, each fixed segment 114 respectively has an element embedding surface 114a, such as a surface with a recess or an opening. The recess or opening is used for embedding into each mechanomyography sensing element 120, so that the mechanomyography sensing element 120 is sealed by a molding compound or adhered in the fixed segments 114 by a tape. In addition, the element embedding surfaces 114a are substantially located on the inner surface of the circular body 110. Thus, when the circular body 110 is mounted on the measuring portion 20 of the testing body 10, the element embedding surfaces 114a of the fixed segments 114 are located at different measuring portions 20 of the testing body 10, so that each mechanomyography sensing element 120 substantially contacts the muscle groups of each measuring portion 20 for measuring the MMG signal of each muscle group.

Referring to FIGS. 1 and 2. In an embodiment, each mechanomyography sensing element 120 can sense the vibration generated when its corresponding muscle group stretches or contracts. Thus, when a palm is bent upward or downward, the vibrations generated by the muscle groups at the top and the bottom of an arm can be sensed by the mechanomyography sensing elements 120 at the top and the bottom. Moreover, when the palm moves to the left and the right, the vibrations generated by the muscle groups at the two sides of the arms can be sensed by the mechanomyography sensing elements 120 at the two sides. Additionally, more dedicated limb movements (such as finger pressing, grasping, pushing, and rotating by the palm or series of movements) can also be detected by more mechanomyography sensing elements 120 according to the vibrations generated by the muscle groups corresponding to an individual finger movement, so that the detectable limb movements are diversified.

Referring to FIGS. 1 and 2, the MMG signal input device 100 further includes a signal processing unit 130. The signal processing unit 130 is such as an embedded chip set which receives an MMG signal Vs of each muscle groups and further integrates the MMG signal Vs to perform front end signal processing (such as noise filtering or signal amplification of a particular wave band), and perform necessary analog-digital conversion so as to transmit the processed MMG signal Vs to a calculating unit 140. The calculating unit 140, realized by such as a computer or a host with sufficient computation capability, receives the MMG signal Vs from the signal processing unit 130, and calculates the feature vector according to the MMG signal Vs to train and create a motion mode. After the motion model is created, the user's input movement can follow the above path, so that the MMG signal Vs again enters the calculating unit 140, which further performs motion recognition to output a control signal Vc. In addition, the control signal Vc is displayed on a screen of a display device 142 to assist the user in confirming the movement corresponding to the inputted MMG signal Vs.

In an embodiment, the mechanomyography sensing element 120 is realized by such as an accelerometer array or a similar acceleration sensing device, wherein the sampling cycle of the mechanomyography sensing element 120 and the measured strength of the signal are already normalized. When each mechanomyography sensing element 120 respectively captures an acceleration in the X-Y-Z axial directions, the signal can respectively be high-pass or low-pass filtered to avoid noise interference.

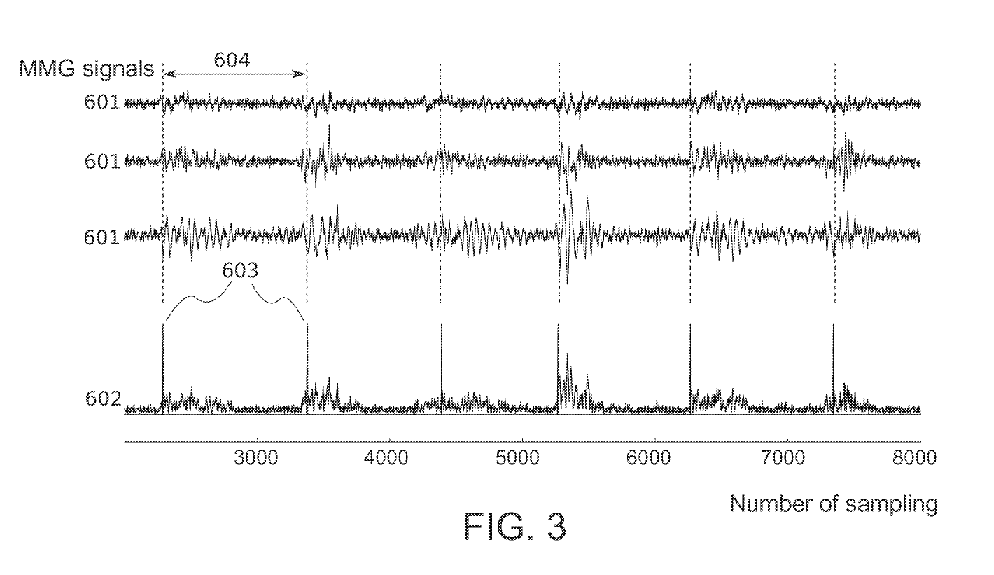

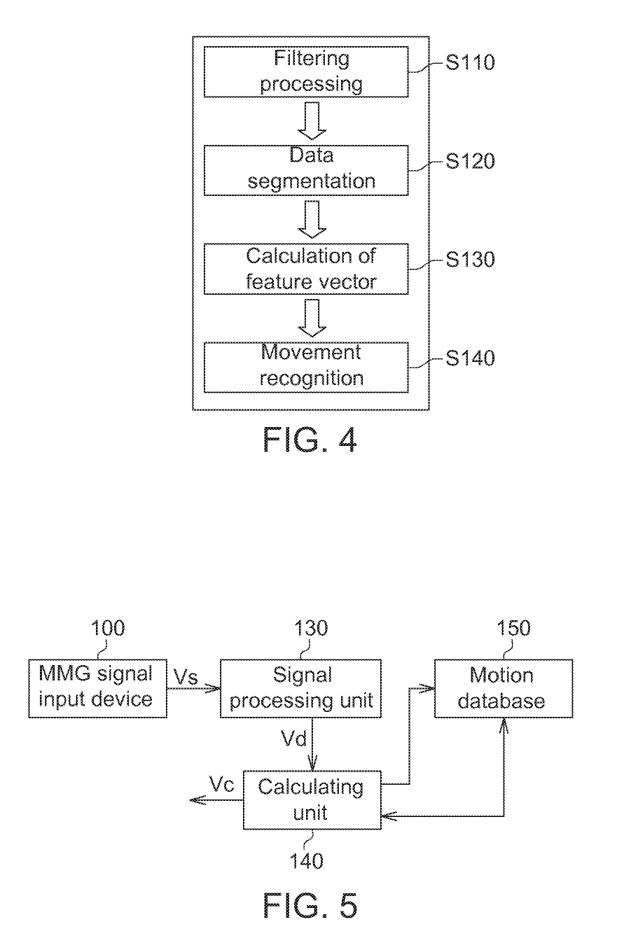

Referring to FIGS. 3, 4 and 5. FIG. 3 shows a measurement diagram of an MMG signal in tri-axial directions according to an embodiment. FIG. 4 shows a processing flowchart of an MMG signal according to an embodiment. FIG. 5 shows a circuit block diagram of a human-machine operating system according to an embodiment. The human-machine operating system includes an MMG signal input device 100, a signal processing unit 130, a calculating unit 140 and a motion database 150. The operation method of the human-machine operating system of FIG. 5 is exemplified below with the MMG signal of FIG. 3 and the process flowchart of the MMG signal of FIG. 4.

As illustrated in FIG. 3, an array accelerometer composed of four mechanomyography sensing elements 120 is taken for example. The MMG signal Vs can be obtained from the acceleration measured by individual accelerometer. Then, the MMG signal Vs is pre-processed by the signal processing unit 130 to obtain a processed signal Vd, which is further transmitted to the calculating unit 140. Then, as indicated in step S110 of FIG. 4, a filtering process is performed on the processed signal Vd to obtain the strength of an acceleration vector g(t). The strengths of the signals in the tri-axial directions can be expressed as:

$$g [ t ] = {\sum }_{n = 0}^{3} ‖ {X}_{H}^{n} [ t ] ‖$$

Wherein, X_{H}[t] denotes a high-pass filtered acceleration vector in tri-axial directions, and n denotes the number of mechanomyography sensing elements. As illustrated in FIG. 3, 601 denotes a tri-axial acceleration value; 602 denotes the strength obtained according to the tri-axial acceleration value 601; 603 denotes the peak obtained from peak measurement according to the strength 602; 604 denotes a segment between two adjacent peaks. According to the peak measurement method, data segmentation is performed to the processed signal Vd as illustrated in step S120 to obtain a segmented data, so that the testing body's complete movement signal can be singulated.

In step S130, feature vector calculation is performed on the segment data to obtain a feature vector data. The calculating unit 140 calculates the characteristic values of the feature vector data such as the mean, the standard deviation and the absolute summation of the segment data so as to perform motion recognition as illustrated in step S140. The motion database 150 is sued for storing a pre-created motion mode data. In an embodiment, the calculating unit 140 can train the feature vector data by support vector machine (SVM) method to create a complete motion mode. The motion mode, after having been created, is stored in the motion database 150 via the motion mode created by the calculating unit 140 and used as a reference for the subsequent motion recognition of the testing body.

Referring to step S140. In an embodiment, when motion recognition is tested, the calculating unit 140, according to at least one of the feature vector data (such as the mean, the standard deviation and/or the absolute summation), performs the motion mode of recognizing the motion of the testing body with the motion mode data stored in the motion database 150. In an embodiment, the feature vector data and the motion mode can be trained again by the SVM method to update the motion mode previously stored in the motion database 150. The calculating unit 140 outputs a control signal Vc corresponding to the movement according to the result of recognition. The control signal Vc is such as a direction signal, an amplification signal, reduction signal, a rotation signal, a click signal or a scrolling signal for controlling a man-machine operation interface such as a cursor, a direction key or a working window. Thus, the human-machine operating system of the present embodiment is capable of recognizing and transforming the MMG signal into a control signal Vc of different information for the user to control a peripheral device.

According to the mechanomyography (MMG) signal input device, the human-machine operating system and the identification method thereof disclosed in the above embodiments of the disclosure, a movement generated when the muscle groups of the limbs collaboratively stretch or contract is used as an input signal, and the feature vector is calculated and the movement corresponding to the MMG signals of the muscle groups is recognized through signal processing so as to output a control signal.

The present embodiment discloses the following features:

(1) The elastic segments of the circular body are made from a flexible or an elastic material, so that a plurality of mechanomyography sensing elements can be mounted on the measuring portion and tightly appressed on the testing body's skin surface for increasing the accuracy of detecting the MMG signals.

(2) The fixed segments of the circular body can firmly position a plurality of mechanomyography sensing elements at the measuring portion for the convenience of the user's operation.

(3) An MMG signal is detected by the MMG signal input device for outputting a control signal to replace conventional input device, so as to provide a man-machine operation interface with fewer restrictions but higher interaction to those users who are incapable of using conventional input device due to broken fingers or palms, abnormal upper limbs or other factors (such as the restriction in space, facility, operating characteristics) to bring about more choice to the users.

While the disclosure has been described by way of example and in terms of the exemplary embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A mechanomyography (MMG) signal input device, comprising:
a circular body having a plurality of elastic segments and fixed segments interlaced and sequentially connected into one piece, wherein the lengths of the elastic segments are adjustable, so that the circular body is mounted on a measuring portion of a testing body, the fixed segments respectively have an element embedding surface, and the measuring portion has a plurality of muscle groups; and
a plurality of mechanomyography sensing elements disposed on the element embedding surface for substantially contacting the measuring portion and respectively measuring MMG signals of the muscle groups.

2. The MMG signal input device according to claim 1, further comprising a signal processing unit for receiving the MMG signals of the muscle groups.

3. The MMG signal input device according to claim 1, wherein the mechanomyography sensing elements comprise an accelerometer array.

4. A human-machine operating system, comprising:
an MMG signal input device mounted on a measuring portion of a testing body, wherein the measuring portion has a plurality of muscle groups;
a signal processing unit for receiving the MMG signals of the muscle groups, and performing integration and pre-processing on the MMG signals to obtain a processed signal;
a motion database for storing a motion mode; and
a calculating unit for receiving the processed signal and performing signal intensity computation and data segmentation to obtain a segment data, performing feature vector calculation on the segment data to obtain a feature vector data, and performing motion recognition on the testing body according to the feature vector data and the motion mode to output a corresponding control signal.

5. The human-machine operating system according to claim 4, wherein the calculating unit further performs training on the feature vector data by a support vector machine (SVM) method to create and store the motion mode in the motion database.

6. The human-machine operating system according to claim 4, wherein the calculating unit further performs training on the feature vector data and the motion mode by the support vector machine (SVM) method to update the motion mode previously stored in the motion database.

7. The human-machine operating system according to claim 4, wherein the calculating unit further performs data segmentation by a peak measurement method to obtain the segment data.

8. The human-machine operating system according to claim 4, wherein the MMG signal input device comprises:
a circular body having a plurality of elastic segments and fixed segments interlaced and sequentially connected into one piece, wherein the lengths of the elastic segments are adjustable, so that the circular body is mounted on the measuring portion of the testing body, and the fixed segments respectively have an element embedding surface; and
a plurality of mechanomyography sensing elements disposed on the element embedding surfaces for substantially contacting the measuring portion and respectively measuring the MMG signals of the muscle groups.

9. The human-machine operating system according to claim 8, wherein the mechanomyography sensing elements comprise an accelerometer array.

10. An MMG signal identification method, comprising:
receiving a plurality of MMG signals generated when a plurality of muscle groups of a testing body stretch or contract;
performing signal integration and pre-processing according to the MMG signals to obtain a processed signal;
performing signal intensity computation and data segmentation according to the processed signal to obtain a segment data, and performing feature vector calculation on the segment data to obtain a feature vector data;
performing motion recognition on the testing body according to the feature vector data and a motion mode; and
outputting a control signal according to a result of the motion recognition.

11. The MMG signal identification method according to claim 10, wherein the feature vector data is trained by a support vector machine (SVM) method to create and store the motion mode in a motion database.

12. The MMG signal identification method according to claim 11, wherein the feature vector data and the motion mode are trained again by the SVM method to update the motion mode previously stored in the motion database.

13. The MMG signal identification method according to claim 10, wherein the feature vector data comprises mean, standard deviation and absolute summation of the segment data.

14. The MMG signal identification method according to claim 10, wherein data segmentation is performed by a peak measurement method to obtain the segment data.
